(19) United States
(12) Patent Application Publication
(10) Pub. No.: US 2021/0038558 A1
(43) Pub. Date: Feb. 11, 2021

(54) WATER-SOLUBLE CANNABINOIDS

(71) Applicant: Orochem Technologies Inc., Naperville, IL (US)

(72) Inventors: Babu Siddegowda Antharavally, Caledonia, IL (US); Anil Rajaram OROSKAR, Oak Brook, IL (US); Pulak SHARMA, Aurora, CO (US); Asha Anil OROSKAR, Oak Brook, IL (US)

(73) Assignee: Orochem Technologies Inc., Naperville, IL (US)

(21) Appl. No.: 16/534,832

(22) Filed: Aug. 7, 2019

(51) Int. Cl. A61K 31/352; A61K 47/26; A61K 47/14; A61K 9/08; A61K 9/10; A61K 9/00

(52) U.S. Cl. CPC A61K 31/352; A61K 47/26; A61K 9/0053; A61K 9/08; A61K 9/10; A61K 47/14

(57) ABSTRACT

Provided is a composition comprising: (a) a polysorbate, (b) vitamin E TPGS, and (c) a cannabinoid. Also provided is a use of the composition for oral consumption and a method of making the composition.

### BACKGROUND

Cannabinoids are a group of compounds originally found and isolated from cannabis or hemp. The terms “hemp” and “cannabis” refer to the genus Cannabis, which contains three species Cannabis sativa, Cannabis indica, and Cannabis ruderalis. All three species are of the family Cannabaceae, which also includes the genus Humulus, or hops. More than 100 different cannabinoids have been isolated including tetrahydrocannabinol (THC), cannabidiol (CBD), and cannabinol (CBN). The discovery of these compounds has led to further discovery of an important neurotransmitter system called the endocannabinoid system. The endocannabinoid system is widely distributed in the brain, and is considered to be responsible for many important bodily functions.

THC is the primary psychoactive component of cannabis and has well known effects on pain, appetite enhancement, digestion, emotions and processes that are mediated through the endocannabinoid system. Contrary to THC, CBD does not appear to have psychoactive properties. However, CBD has been shown to have pharmacological effects in various models of pathologies, from inflammatory and neurodegenerative diseases, to epilepsy, autoimmune disorders like multiple sclerosis, arthritis, schizophrenia, and cancer.

Cannabinoids such as CBD and THC have a strong preference for non-aqueous media, dissolving only in oil and organic solvents, such as alcohol-based solvents, aliphatic hydrocarbons, dimethyl sulfoxide, dimethyl formamide, and acetone. The lack of solubility in water can result in low absorption and bioavailability, thereby presenting a challenge for administration as an orally ingested therapeutic. In order to increase bioavailability and absorption, cannabinoids have been formulated with a number of excipients. However, inclusion of such ingredients can produce cannabinoid compositions with low solubility and stability, and can be costly and difficult to manufacture.

Therefore, there is a need for new formulations for oral consumption of cannabinoids such as CBD and THC that have improved absorption and bioavailability, which are relatively easy to manufacture, and remain shelf stable for extended periods of time.

It will be appreciated that this background description has been created by the inventors to aid the reader, and is not to be taken as a reference to prior art nor as an indication that any of the indicated problems were themselves appreciated in the art. While the described principles can, in some regards and embodiments, alleviate the problems inherent in other systems, it will be appreciated that the scope of the protected innovation is defined by the attached claims, and not by the ability of the claimed invention to solve any specific problem noted herein.

### BRIEF SUMMARY

The present invention is premised, at least in part, on using polysorbate(s) and D-α-Tocopherol polyethylene glycol 1000 succinate (vitamin E TPGS) based formulations to prepare water-soluble cannabinoids. In various embodiments, the invention provides compositions (e.g., compositions for oral consumption) such as solid, liquid, or oil preparations, methods of preparing the compositions, and use of the composition for oral consumption. The compositions comprise a polysorbate, vitamin E TPGS, and a cannabinoid (e.g., CBD or THC). The oral compositions can be non-alcoholic or alcoholic (i.e., additionally containing ethanol, if desired). The present inventors have found a preferred desired proportion of the polysorbate, vitamin E TPGS, and cannabinoid to produce a desirably high concentration of water solubility, while maintaining a high level of shelf stability.

Thus, in one aspect, the invention provides a composition (e.g., solid, liquid, or oil) comprising, consisting essentially of, or consisting of (a) a polysorbate, (b) vitamin E TPGS, and (c) a cannabinoid.

In another aspect, the invention also provides a composition (e.g., in liquid form) comprising, consisting essentially of, or consisting of (a) a polysorbate, (b) vitamin E TPGS, (c) a cannabinoid (e.g., CBD or THC), and (d) water.

In another aspect, the invention provides a composition (e.g., solid, liquid, or oil) comprising, consisting essentially of, or consisting of (a) from about 30 wt. % to about 50 wt. % of a polysorbate, (b) from about 10 wt. % to about 30 wt. % of vitamin E TPGS, and (c) from about 20 wt. % to about 60 wt. % of a cannabinoid (e.g., CBD or THC), wherein the weight percentage is based on the sum total of (a), (b), and (c).

In another aspect, the invention also provides a composition (e.g., in liquid form) comprising, consisting essentially of, or consisting of (a) from about 30 wt. % to about 50 wt. % of a polysorbate, (b) from about 10 wt. % to about 30 wt. % of vitamin E TPGS, (c) from about 20 wt. % to about 60 wt. % of a cannabinoid (e.g., CBD or THC), and (d) water, wherein the weight percentage is based on the sum total of (a), (b), and (c).

In another aspect, the invention also provides a use of a composition described herein for oral consumption.

In another aspect, the invention further provides a method of making a composition for oral consumption comprising: (a) a polysorbate, (b) vitamin E TPGS, and (c) a cannabinoid, the method comprising: preparing an aqueous solution comprising the polysorbate, vitamin E TPGS, and the cannabinoid, and filtering the aqueous solution to form a filtered aqueous solution.

### BRIEF DESCRIPTION OF THE DRAWINGS

FIG. 1 is a high performance liquid chromatography trace of a diluted sample of a diluted composition containing polysorbate 20, vitamin E TPGS, and cannabidiol, as set forth in Example 1.

FIG. 2 is a graph of CBD concentration (Y-axis) vs. temperature (X-axis) for the stability analysis after 84 days of a concentrated composition containing polysorbate 20, vitamin E TPGS, and cannabidiol, as set forth in Example 2.

FIG. 3 is a graph of CBD concentration (Y-axis) vs. temperature (X-axis) for the stability analysis after 35 days of a diluted composition containing polysorbate 20, vitamin E TPGS, and cannabidiol, as set forth in Example 3.

FIG. 4 is a graph of CBD concentration (Y-axis) vs. temperature (X-axis) for the stability analysis after 8 days of a concentrated composition containing polysorbate 20, polysorbate 40, vitamin E TPGS, and cannabidiol, as set forth in Example 5.

FIG. 5 is a graph of CBD concentration (Y-axis) vs. temperature (X-axis) for the stability analysis after 8 days of a diluted composition containing polysorbate 20, polysorbate 40, vitamin E TPGS, and cannabidiol, as set forth in Example 6.

FIG. 6 is a graph of CBD concentration (Y-axis) vs. temperature (X-axis) for the stability analysis after 15 days of a concentrated composition containing polysorbate 20, polysorbate 60, vitamin E TPGS, and cannabidiol, as set forth in Example 8.

FIG. 7 is a graph of CBD concentration (Y-axis) vs. temperature (X-axis) for the stability analysis after 15 days of a diluted composition containing polysorbate 20, polysorbate 60, vitamin E TPGS, and cannabidiol, as set forth in Example 9.

FIG. 8 is a high performance liquid chromatography trace of a diluted sample of a diluted composition containing polysorbate 20, polysorbate 80, vitamin E TPGS, and cannabidiol, as set forth in Example 10.

FIG. 9 is a graph of CBD concentration (Y-axis) vs. temperature (X-axis) for the stability analysis after 19 days of a concentrated composition containing polysorbate 20, polysorbate 80, vitamin E TPGS, and cannabidiol, as set forth in Example 11.

FIG. 10 is a graph of CBD concentration (Y-axis) vs. temperature (X-axis) for the stability analysis after 19 days of a diluted composition containing polysorbate 20, polysorbate 80, vitamin E TPGS, and cannabidiol, as set forth in Example 12.

### DETAILED DESCRIPTION

Embodiments of the invention provide for a composition (e.g., solid, liquid, or oil) containing a cannabinoid, which can be used for oral consumption (e.g., as a liquid preparation), and a method of making the composition containing the cannabinoid. Advantageously, compositions in accordance with preferred embodiments of the invention exhibit enhanced stability for extended periods of time. In addition, compositions for oral consumption in accordance with preferred embodiments of the invention can exhibit improved absorption and bioavailability. Thus, embodiments of the invention represent improvements to conventional techniques. In this respect, as described herein, cannabinoids such as THC and CBD can be insoluble in water and therefore require additional components to facilitate solubilization (“solubilizing agents”). In order to achieve desirable levels of said cannabinoids in an aqueous solution, and to maintain said desirable levels, compositions and methods have been developed as described herein. Other benefits of the inventive process and compositions will be readily apparent from the disclosure provided herein.

Embodiments of the invention provide a composition comprising: (a) a polysorbate, (b) vitamin E TPGS, and (c) a cannabinoid.

The composition comprises a polysorbate. As used herein, the term polysorbate refers to a class of emulsifiers derived from sorbitol. The polysorbate can be any suitable polysorbate with any suitable molecular weight. Accordingly, in various embodiments, the polysorbate can have a weight average molecular weight of, for example, from about 1000 g/mol to about 1400 g/mol (e.g., from about 1100 g/mol to about 1400 g/mol, from about 1150 g/mol to about 1400 g/mol, from about 1200 g/mol to about 1400 g/mol, from about 1150 g/mol to about 1350 g/mol, from about 1150 g/mol to about 1300 g/mol, from about 1200 g/mol to about 1400 g/mol, from about 1200 g/mol to about 1350 g/mol, from about 1200 g/mol to about 1300 g/mol, or from about 1250 g/mol to about 1400 g/mol).

In some embodiments, the polysorbate is polysorbate 20 (polyoxyethylene (20) sorbitan monolaurate), polysorbate 40 (polyoxyethylene (20) sorbitan monopalmitate), polysorbate 60 (polyoxyethylene (20) sorbitan monostearate), polysorbate 80 (polyoxyethylene (20) sorbitan monooleate), or a combination thereof. While not wishing to be bound by any particular theory, it is believed that in terms of composition stability, polysorbate 20 is superior to polysorbate 40, polysorbate 40 is superior to polysorbate 60, and polysorbate 60 is superior to polysorbate 80.

In certain embodiments, the composition comprises polysorbate 20. Accordingly, the polysorbate can be polysorbate 20 or the polysorbate can contain polysorbate 20 and one or more of polysorbate 40, polysorbate 60, and polysorbate 80. In other embodiments, the composition comprises polysorbate 40. Accordingly, the polysorbate can be polysorbate 40 or the polysorbate can be polysorbate 40 and one or more of polysorbate 20, polysorbate 60, and polysorbate 80.

In preferred embodiments, the composition comprises polysorbate 20; polysorbate 20 and polysorbate 40; polysorbate 20 and polysorbate 60; or polysorbate 20 and polysorbate 80.

The composition can comprise any suitable amount of polysorbate (i.e., sum total of polysorbates). For example, the composition can comprise from about 5 wt. % to about 50 wt. % of the polysorbate (i.e., sum total of polysorbates), for example, from about 10 wt. % to about 50 wt. %, from about 15 wt. % to about 50 wt. %, from about 20 wt. % to about 50 wt. %, from about 25 wt. % to about 50 wt. %, from about 30 wt. % to about 50 wt. %, from about 35 wt. % to about 45 wt. %, from about 5 wt. % to about 45 wt. %, from about 10 wt. % to about 45 wt. %, from about 15 wt. % to about 45 wt. %, from about 20 wt. % to about 45 wt. %, from about 25 wt. % to about 50 wt. %, from about 30 wt. % to about 45 wt. %, from about 35 wt. % to about 45 wt. %, from about 5 wt. % to about 40 wt. %, from about 10 wt. % to about 40 wt. %, from about 15 wt. % to about 40 wt. %, from about 20 wt. % to about 40 wt. %, from about 25 wt. % to about 40 wt. %, from about 30 wt. % to about 40 wt. %, or from about 35 wt. % to about 40 wt. % of the polysorbate (i.e., sum total of polysorbates), wherein the wt. % is based on the sum total of ingredients other than water. In some embodiments, the composition comprises from about 30 wt. % to about 50 wt. % of the polysorbate (i.e., sum total of polysorbates), wherein the wt. % is based on the sum total of ingredients other than water. In certain embodiments, the composition comprises from about 35 wt. % to about 45 wt. % of the polysorbate, wherein the wt. % is based on the sum total of ingredients other than water.

The composition comprises vitamin E TPGS. The composition can comprise any suitable amount of vitamin E TPGS. For example, the composition can comprise from about 1 wt. % to about 50 wt. % of vitamin E TPGS, for example, from about 5 wt. % to about 50 wt. %, from about 10 wt. % to about 50 wt. %, from about 15 wt. % to about 50 wt. %, from about 20 wt. % to about 50 wt. %, from about 25 wt. % to about 50 wt. %, from about 30 wt. % to about 50 wt. %, from about 1 wt. % to about 40 wt. %, from about 5 wt. % to about 40 wt. %, from about 10 wt. % to about 40 wt. %, from about 15 wt. % to about 40 wt. %, from about 20 wt. % to about 40 wt. %, from about 25 wt. % to about 40 wt. %, from about 30 wt. % to about 40 wt. %, from about 1 wt. % to about 30 wt. %, from about 5 wt. % to about 30 wt. %, from about 10 wt. % to about 30 wt. %, from about 15 wt. % to about 30 wt. %, from about 20 wt. % to about 30 wt. %, from about 25 wt. % to about 30 wt. %, or from about 15 wt. % to about 25 wt. % of vitamin E TPGS, wherein the wt. % is based on the sum total of ingredients other than water. In some embodiments, the composition comprises from about 10 wt. % to about 30 wt. % of vitamin E TPGS, wherein the wt. % is based on the sum total of ingredients other than water. In certain embodiments, the composition comprises from about 15 wt. % to about 25 wt. % of vitamin E TPGS, wherein the wt. % is based on the sum total of ingredients other than water.

The composition comprises a cannabinoid. The cannabinoids can be isolated from plants (e.g., dried hemp and/or cannabis leaves) of the genus Cannabis, which contains three species, namely Cannabis sativa, Cannabis indica, and Cannabis ruderalis. The isolated extract can be used directly, or the isolated extract can be purified (e.g., using column chromatography) and/or processed (e.g., filtered, dewaxed, decolorized, and/or decarboxylated) prior to use in the composition. Accordingly, the cannabinoid can be a single purified cannabinoid (e.g., CBD or THC), a mixture of cannabinoids, or an extract from a Cannabis plant. The cannabinoid can be any cannabinoid isolated from the Cannabis plant.

The following are commonly found cannabinoids in the extract of hemp leaves, which can be used in the compositions described herein:

| | |
|---|---|
| THC | Tetrahydrocannabinol |
| THCV | Tetrahydrocannabivarin |
| CBG | Cannabigerol |
| CBD | Cannabidiol |
| CBC | Cannabichromene |
| CBN | Cannabinol |
| THCA | Tetrahydrocannabinolic Acid |
| CBDA | Cannabidiolic Acid |
| CBGA | Cannabigerolic Acid |
| CBDV | Cannabidivarin |

Accordingly, the cannabinoid can be THC, THCV, CBG, CBD, CBC, CBN, THCA, CBDA, CBGA, CBDV, or a combination thereof.

In some embodiments, the cannabinoid is cannabidiol (CBD) and/or tetrahydrocannabinol (THC). In certain embodiments, the cannabinoid (e.g., CBD and/or THC) is purified to high levels (e.g., greater than 90% purity, greater than 95% purity, or greater than 99% purity) for use in the composition, thereby allowing for their use in various pharmaceutical and nutraceutical applications. In some embodiments, the cannabinoid is THC. In preferred embodiments, the cannabinoid is CBD. In certain aspects purified CBD can be used, which has the benefits of CBD without the alternative effects of psychoactive THC.

The composition can comprise any suitable amount of the cannabinoid (i.e., sum total of cannabinoids). For example, the composition can comprise from about 1 wt. % to about 70 wt. % of the cannabinoid (i.e., sum total of cannabinoids), for example, from about 5 wt. % to about 70 wt. %, from about 10 wt. % to about 70 wt. %, from about 15 wt. % to about 70 wt. %, from about 20 wt. % to about 70 wt. %, from about 25 wt. % to about 70 wt. %, from about 30 wt. % to about 70 wt. %, from about 1 wt. % to about 60 wt. %, from about 5 wt. % to about 60 wt. %, from about 10 wt. % to about 60 wt. %, from about 15 wt. % to about 60 wt. %, from about 20 wt. % to about 60 wt. %, from about 25 wt. % to about 60 wt. %, from about 30 wt. % to about 60 wt. %, from about 1 wt. % to about 50 wt. %, from about 5 wt. % to about 50 wt. %, from about 10 wt. % to about 50 wt. %, from about 15 wt. % to about 50 wt. %, from about 20 wt. % to about 50 wt. %, from about 25 wt. % to about 50 wt. %, or from about 30 wt. % to about 50 wt. % of the cannabinoid (i.e., sum total of cannabinoids), wherein the wt. % is based on the sum total of ingredients other than water. In some embodiments, the composition comprises from about 20 wt. % to about 60 wt. % of the cannabinoid (i.e., sum total of cannabinoids), wherein the wt. % is based on the sum total of ingredients other than water. In certain embodiments, the composition comprises from about 30 wt. % to about 50 wt. % of the cannabinoid (i.e., sum total of cannabinoids), wherein the wt. % is based on the sum total of ingredients other than water.

In some embodiments, the composition comprises from about 30 wt. % to about 50 wt. % of a polysorbate (i.e., sum total of polysorbates), from about 10 wt. % to about 30 wt. % of vitamin E TPGS, and from about 20 wt. % to about 60 wt. % of a cannabinoid (i.e., sum total of cannabinoids), wherein the weight percentage is based on the sum total of the polysorbate (i.e., sum total of polysorbates), vitamin E TPGS, and the cannabinoid (i.e., sum total of cannabinoids). In certain embodiments, the composition comprises from about 35 wt. % to about 45 wt. % of a polysorbate (i.e., sum total of polysorbates), from about 15 wt. % to about 25 wt. % of vitamin E TPGS, and from about 30 wt. % to about 50 wt. % of a cannabinoid (i.e., sum total of cannabinoids), wherein the weight percentage is based on the sum total of the polysorbate (i.e., sum total of polysorbates), vitamin E TPGS, and the cannabinoid (i.e., sum total of cannabinoids).

The composition can be a solid, oil, or aqueous solution. Accordingly, the compositions described herein can further comprises water. The water can be any type of water suitable for oral consumption. For example, the water can be purified (e.g., by filtration, distillation, or reverse osmosis), carbonated, dyed, flavored, or any combination thereof. When the composition is an aqueous solution, the aqueous solution can have a cannabinoid (e.g., CBD and/or THC) concentration of from about 0.05 mg/mL to about 20 mg/mL, from about 0.05 mg/mL to about 10 mg/mL, from about 0.05 mg/mL to about 5 mg/mL, or from about 0.05 mg/mL to about 1 mg/mL.

The concentration of cannabinoid in the composition can be measured by any suitable method. In some embodiments, the concentration of cannabinoid in the composition is analyzed by high-performance liquid chromatography (“HPLC”). A skilled artisan will know suitable parameters for analyzing the composition by HPLC, such that the amounts of the cannabinoid in the composition can be determined.

In some embodiments, the compositions described herein are shelf stable for an extended period of time. For example, the compositions described herein can be shelf stable for at least 1 month, at least 3 months, at least 6 months, at least 1 year, at least 2 years, or at least 5 years. As used herein, the phrase “shelf stable” refers to a composition, which is enclosed in an aluminum, plastic, or glass container, that maintains at least about 90% of the cannabinoid concentration of the original composition (e.g., at least about 95% of the cannabinoid concentration of the original composition) over the course of the shelf life. In certain embodiments, the composition is shelf stable at 4° C. for at least 3 months (e.g., at least 6 months, at least 1 year, at least 2 years, or at least 5 years). In other embodiments, the composition is shelf stable at 23° C. for at least 3 months (e.g., at least 6 months, at least 1 year, at least 2 years, or at least 5 years). In some embodiments, the composition is shelf stable at 37° C. for at least 3 months (e.g., at least 6 months, at least 1 year, at least 2 years, or at least 5 years). The stability of the composition also can be monitored qualitatively by color. Without wishing to be bound by any particular theory, it is believed that the cannabinoids can be oxidized over time, which in turn leads to the solution turning yellow. The compositions described herein aim to reduce the foregoing oxidation process, thereby reducing the change in color.

The composition can further comprise one or more pharmaceutically acceptable excipients. Suitable excipients and the amounts to use may be readily determined under the direction of one of ordinary skill in the art based upon experience and consideration of standard procedures and reference works in the field, e.g., sweetening agents, dyes, flavoring agents, and preservatives.

The compositions described herein can be used for oral consumption. For example, the compositions can be used in food (e.g., cooking ingredients, baked goods, energy bars, etc.) and beverages (e.g., energy drinks, hydration beverages, alcoholic beverages, etc.). In some embodiments, particularly when the cannibinoid is free of THC (e.g., it contains CBD), the composition is an alcoholic beverage for oral consumption. Accordingly, in some embodiments, the compositions described herein can further comprise ethanol.

For compositions comprising ethanol, the composition can comprise ethanol in an amount from about 5 mL to about 50 mL per 100 mL of water. For example, the composition can comprise ethanol in an amount from about 5 mL to about 40 mL per 100 mL of water, from about 5 mL to about 30 mL per 100 mL of water, from about 5 mL to about 20 mL per 100 mL of water, from about 10 mL to about 50 mL per 100 mL of water, from about 10 mL to about 40 mL per 100 mL of water, from about 10 mL to about 30 mL per 100 mL of water, or from about 10 mL to about 20 mL per 100 mL of water.

When the composition consists essentially of certain ingredients, other components that exert a material effect (e.g., modify the solubility or stability of the cannabinoid) are excluded from the composition, with the exception of trace amounts of water and/or ethanol. When the composition for oral consumption consists of certain ingredients, the composition excludes any other components, including water and/or ethanol, if not explicitly included.

The invention further provides a method of making a composition for oral consumption comprising: (a) a polysorbate, (b) vitamin E TPGS, and (c) a cannabinoid, the method comprising: preparing an aqueous solution comprising the polysorbate, vitamin E TPGS, and the cannabinoid, and filtering the aqueous solution to form a filtered aqueous solution. The final concentration of (a) a polysorbate, (b) vitamin E TPGS, and (c) a cannabinoid in the composition are consistent with the values described herein.

The aqueous solution can be filtered by any suitable technique to remove residual particulates in the aqueous solution. In certain embodiments, the aqueous solution is filtered with a micro filter. For example, the aqueous solution can be filtered using a filter (e.g., a microfiber filter) with a particle retention of at least about 0.1 μm (e.g., at least about 0.2 μm, at least about 0.3 μm, at least about 0.4 μm, at least about 0.5 μm, at least about 0.6 μm, at least about 0.7 μm, at least about 0.8 μm, at least about 0.9 μm, or at least about 1 μm). In some embodiments, the aqueous solution is be filtered with a Whatman™ glass microfiber filter and/or a polyethersulfone (PES) filter. In some embodiments, the aqueous solution is filtered using a 0.7 μm Whatman™ glass microfiber filter, a 0.2 μm polyethersulfone (PES) filter, a 0.22 μm polyethersulfone (PES) filter, or a combination thereof.

In some embodiments, the method further comprises diluting the filtered aqueous solution to form a diluted aqueous solution. The filtered aqueous solution can be diluted with water and/or alcohol. Without wishing to be bound by any particular theory, it is believed that the non-diluted composition (i.e., stock solutions) may be too concentrated for oral consumption, and may need to be diluted.

In some embodiments, the method further comprises removing at least a portion of the water from the filtered aqueous solution to form a concentrated aqueous solution. The water can be removed by any suitable method. For example, the water can be removed by evaporation (e.g., under reduced pressure, elevated temperature, or a combination thereof, membrane permeation (e.g., nano-filtration), or a combination thereof. The concentrated aqueous solution can contain water, can be substantially free of water, or can be free of water. Accordingly, the concentrated aqueous solution can be an aqueous solution, an oil, or a solid. The concentrated aqueous solution can be used as a concentrate for storage and transportation purposes. For example, the concentrated aqueous solution can be a powder, which can be packaged and then solubilized in water and/or alcohol (e.g., by a user) to form a composition for oral consumption.

In some embodiments, the method further comprises adding a pharmaceutically acceptable excipient to the aqueous solution, the filtered aqueous solution, the diluted aqueous solution, the concentrated aqueous solution, or a combination thereof.

In some embodiments, the method further comprises adding ethanol to the aqueous solution, the filtered aqueous solution, the diluted aqueous solution, the concentrated aqueous solution, or a combination thereof.

In some embodiments, the method comprises packaging a composition described herein. The composition can be packaged in any suitable container (e.g., aluminum, plastic, glass, cardboard, paper, etc.). The packaged composition can be stored and/or transported at any suitable temperature. In some embodiments, the packaged composition is stored and/or transported at a temperature of about 37° C. or less, e.g., about 30° C. or less, or about 23° C. or less. In preferred embodiments, the packaged composition is stored at a temperature of about 30° C. or less.

The amounts of a polysorbate, vitamin E TPGS, and a cannabinoid (e.g., CBD and/or THC) suitable for the methods described herein will be readily apparent from the disclosure as a whole.

### EMBODIMENTS

Principles of the present disclosure are incorporated in the following, non-limiting examples of embodiments:

Embodiment (1) A composition comprising: (a) a polysorbate, (b) vitamin E TPGS, and (c) a cannabinoid.

Embodiment (2) The composition of embodiment (1), comprising: (a) from about 30 wt. % to about 50 wt. % of the polysorbate, (b) from about 10 wt. % to about 30 wt. % of vitamin E TPGS, and (c) from about 20 wt. % to about 60 wt. % of a cannabinoid, wherein the weight percentage is based on the sum total of (a), (b), and (c).

Embodiment (3) The composition of embodiment (2), comprising: (a) from about 35 wt. % to about 45 wt. % of the polysorbate, (b) from about 15 wt. % to about 25 wt. % of vitamin E TPGS, and (c) from about 30 wt. % to about 50 wt. % of a cannabinoid, wherein the weight percentage is based on the sum total of (a), (b), and (c).

Embodiment (4) The composition of any one of embodiments (1)-(3), wherein the polysorbate is polysorbate 20, polysorbate 40, polysorbate 60, polysorbate 80, or a combination thereof.

Embodiment (5) The composition of any one of embodiments (1)-(4), wherein the polysorbate is polysorbate 20 and one or more of polysorbate 40, polysorbate 60, and polysorbate 80.

Embodiment (6) The composition of any one of embodiments (1)-(4), wherein the polysorbate is polysorbate 20.

Embodiment (7) The composition of any one of embodiments (1)-(4), wherein the polysorbate is polysorbate 40 and one or more of polysorbate 20, polysorbate 60, and polysorbate 80.

Embodiment (8) The composition of any one of embodiments (1)-(4), wherein the polysorbate is polysorbate 40.

Embodiment (9) The composition of any one of embodiments (1)-(8), wherein the cannabinoid is cannabidiol, tetrahydrocannabinol, or a combination thereof.

Embodiment (10) The composition of any one of embodiments (1)-(9), wherein the cannabinoid is cannabidiol.

Embodiment (11) The composition of any one of embodiments (1)-(9), wherein the cannabinoid is tetrahydrocannabinol.

Embodiment (12) The composition of any one of embodiments (1)-(11), wherein the composition is a solid.

Embodiment (13) The composition of any one of embodiments (1)-(11), wherein the composition is an oil.

Embodiment (14) The composition of any one of embodiments (1)-(11), wherein the composition is an aqueous solution.

Embodiment (15) The composition of embodiment (14), wherein the aqueous solution has a cannabinoid concentration of from about 0.05 mg/mL to about 20 mg/mL.

Embodiment (16) The composition of embodiment (15), wherein the aqueous solution has a cannabinoid concentration of from about 0.05 mg/mL to about 10 mg/mL.

Embodiment (17) The composition of embodiment (16), wherein the aqueous solution has a cannabinoid concentration of from about 0.05 mg/mL to about 5 mg/mL.

Embodiment (18) The composition of embodiment (17), wherein the aqueous solution has a cannabinoid concentration of from about 0.05 mg/mL to about 1 mg/mL.

Embodiment (19) The composition of any one of embodiments (1)-(18), wherein the composition further comprises one or more pharmaceutically acceptable excipients.

Embodiment (20) Use of the composition of any one of embodiments (1)-(19) for oral consumption.

Embodiment (21) A method of making a composition for oral consumption comprising: (a) a polysorbate, (b) vitamin E TPGS, and (c) a cannabinoid, the method comprising: preparing an aqueous solution comprising the polysorbate, vitamin E TPGS, and the cannabinoid, and filtering the aqueous solution to form a filtered aqueous solution.

Embodiment (22) The method of embodiment (21), wherein the polysorbate is polysorbate 20, polysorbate 40, polysorbate 60, polysorbate 80, or a combination thereof.

Embodiment (23) The method of embodiment (21) or embodiment (22), wherein the polysorbate is polysorbate 20 and one or more of polysorbate 40, polysorbate 60, and polysorbate 80.

Embodiment (24) The method of embodiment (21) or embodiment (22), wherein the polysorbate is polysorbate 20.

Embodiment (25) The method of embodiment (21) or embodiment (22), wherein the polysorbate is polysorbate 40 and one or more of polysorbate 20, polysorbate 60, and polysorbate 80.

Embodiment (26) The method of embodiment (21) or embodiment (22), wherein the polysorbate is polysorbate 40.

Embodiment (27) The method of any one of embodiments (21)-(26), wherein the cannabinoid is cannabidiol, tetrahydrocannabinol, or a combination thereof.

Embodiment (28) The method of embodiment (27), wherein the cannabinoid is cannabidiol.

Embodiment (29) The method of embodiment (27), wherein the cannabinoid is tetrahydrocannabinol.

Embodiment (30) The method of any one of embodiments (21)-(29), further comprising diluting the filtered aqueous solution to form a diluted aqueous solution.

Embodiment (31) The method of any one of embodiments (21)-(29), further comprising removing at least a portion of the water from the filtered aqueous solution to form a concentrated aqueous solution.

Embodiment (32) The method of embodiment (31), wherein the concentrated aqueous solution is a solid.

Embodiment (33) The method of embodiment (31), wherein the concentrated aqueous solution is an oil.

Embodiment (34) The method of any one of embodiments (21)-(33), further comprising adding a pharmaceutically acceptable excipient to the aqueous solution, the filtered aqueous solution, the diluted aqueous solution, the concentrated aqueous solution, or a combination thereof.

The foregoing exemplary embodiments of the disclosure numbered 1-34 are non-limiting. Other exemplary embodiments are apparent from the entirety of the description herein. As will be apparent to those of skill in the art upon reading this disclosure, each of the individually numbered embodiments may be used or combined with any of the preceding or following individually numbered aspects.

The following examples further illustrate the invention but, of course, should not be construed as in any way limiting its scope.

### Example 1

This example illustrates a method of preparing a water soluble cannabidiol composition containing polysorbate 20, vitamin E TPGS, and cannabidiol.

Polysorbate 20 (15 mL) was added to high performance liquid chromatography (HPLC) water (985 mL) at 50° C. Vitamin E TPGS (7.5 g) was added to the resulting mixture and the mixture was stirred for 30-60 minutes at 50° C. Cannabidiol (15 g) was slowly added at 50° C. and the resulting composition was mixed for 16 hours. The resulting mixture was filtered through a 0.7 μm Whatman™ glass microfiber filter followed by a 0.2 μm polyethersulfone (PES) filter to produce a composition containing 8.7 mg/mL cannabidiol. The composition was diluted 83.33-fold in water and filtered through a 0.22 μm polyethersulfone (PES) filter to produce a composition containing 0.104 mg/mL cannabidiol.

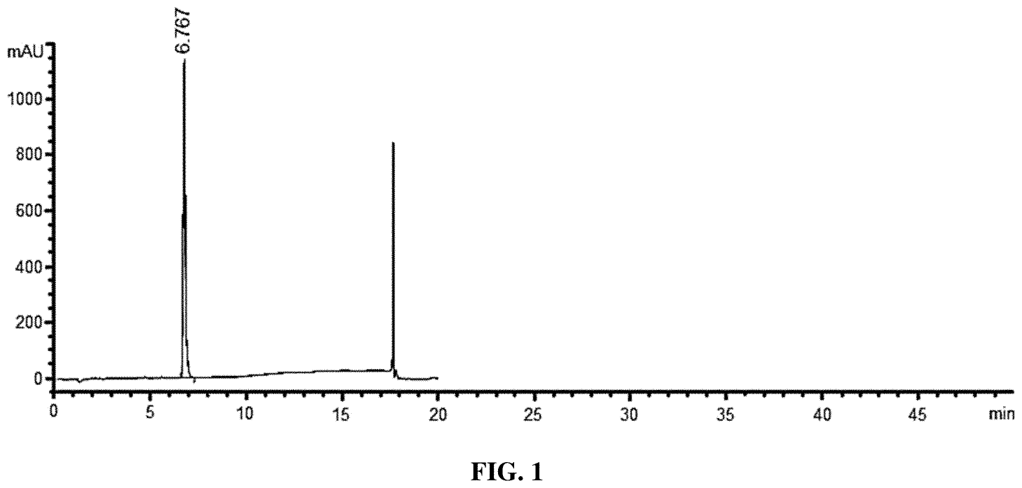

The diluted solution containing polysorbate 20, vitamin E TPGS, and cannabidiol was analyzed using HPLC with the following parameters:
Sample: injected 20 μl
Column: Reliasil, C18, 3 μm, 4.6×150 mm
Temperature: 25° C.
Wavelength: 220 nm
Mobile Phase: A: 0.2% Phosphoric acid in water, B: ACN
Gradient was run from 80:20 (B:A) over 20 min.
   The resulting HPLC trace is set forth in FIG. 1.

### Example 2

This example illustrates the stability of a concentrated composition containing polysorbate 20, vitamin E TPGS, and cannabidiol.

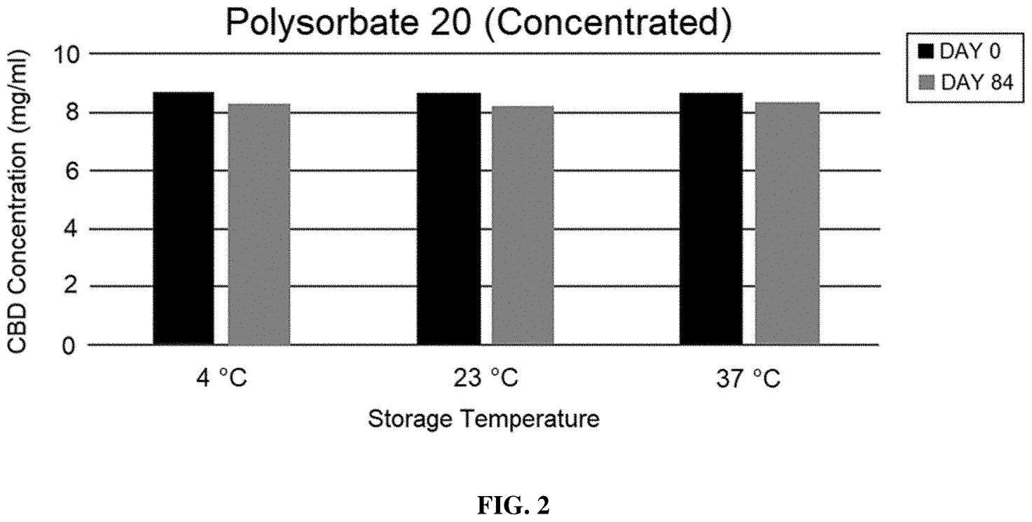

The stock composition from Example 1, containing polysorbate 20, vitamin E TPGS, and 8.7 mg/mL of cannabidiol was stored at three separate temperatures of 4° C., room temperature (i.e., about 23° C.), and 37° C. The stock solution at each one of these temperatures was diluted 83.33 fold in drinking water at day 0 and day 84 and analyzed using the HPLC parameters set forth in Example 1. The results are set forth in FIG. 2.

As is apparent from the results set forth in FIG. 2, the concentrated composition containing polysorbate 20, vitamin E TPGS, and 8.7 mg/mL of cannabidiol was stable, resulting in minimal loss of cannabidiol at temperatures of 4° C., room temperature (i.e., about 23° C.), and 37° C.

### Example 3

This example illustrates the stability of a diluted composition containing polysorbate 20, vitamin E TPGS, and cannabidiol.

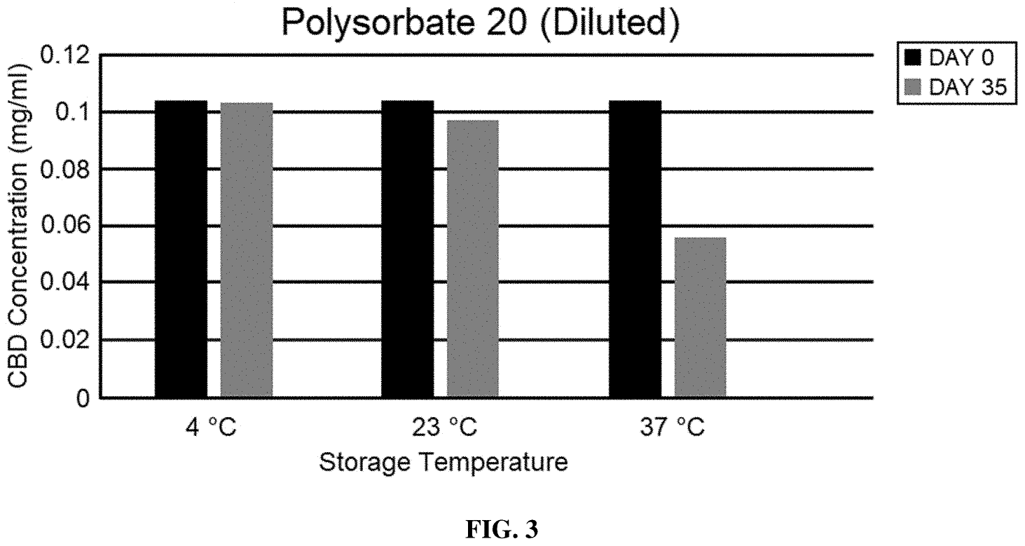

The diluted composition from Example 1, containing polysorbate 20, vitamin E TPGS, and 0.104 mg/mL of cannabidiol was stored at three separate temperatures of 4° C., room temperature (i.e., about 23° C.), and 37° C. The diluted composition at each of these temperatures was analyzed at day 0 and day 35 using the HPLC parameters set forth in Example 1. The results are set forth in FIG. 3.

As is apparent from the results set forth in FIG. 3, the diluted composition containing polysorbate 20, vitamin E TPGS, and 0.104 mg/mL of cannabidiol maintained comparable stability relative to the concentrated composition of Example 2 at temperatures of 4° C. and room temperature (i.e., about 23° C.), resulting in minimal loss of cannabidiol.

### Example 4

This example illustrates a method of preparing a water soluble cannabidiol composition containing polysorbate 20, polysorbate 40, vitamin E TPGS, and cannabidiol.

Polysorbate 20 (7.5 mL) and polysorbate 40 (7.5 mL) were added to high performance liquid chromatography (HPLC) water (985 mL) at 50° C. Vitamin E TPGS (7.5 g) was added to the resulting mixture and the mixture was stirred for 30-60 minutes at 50° C. Cannabidiol (15 g) was slowly added at 50° C. and the resulting composition was mixed for 16 hours. The resulting mixture was filtered through a 0.7 μm Whatman™ glass microfiber filter followed by a 0.2 μm polyethersulfone (PES) filter to produce a composition containing 8.44 mg/mL cannabidiol. The composition was diluted 83.33-fold in water and filtered through a 0.22 μm polyethersulfone (PES) filter to produce a composition containing 0.101 mg/mL cannabidiol. The diluted composition containing 0.101 mg/mL cannabidiol was analyzed using the HPLC parameters set forth in Example 1.

### Example 5

This example illustrates the stability of a concentrated composition containing polysorbate 20, polysorbate 40, vitamin E TPGS, and cannabidiol.

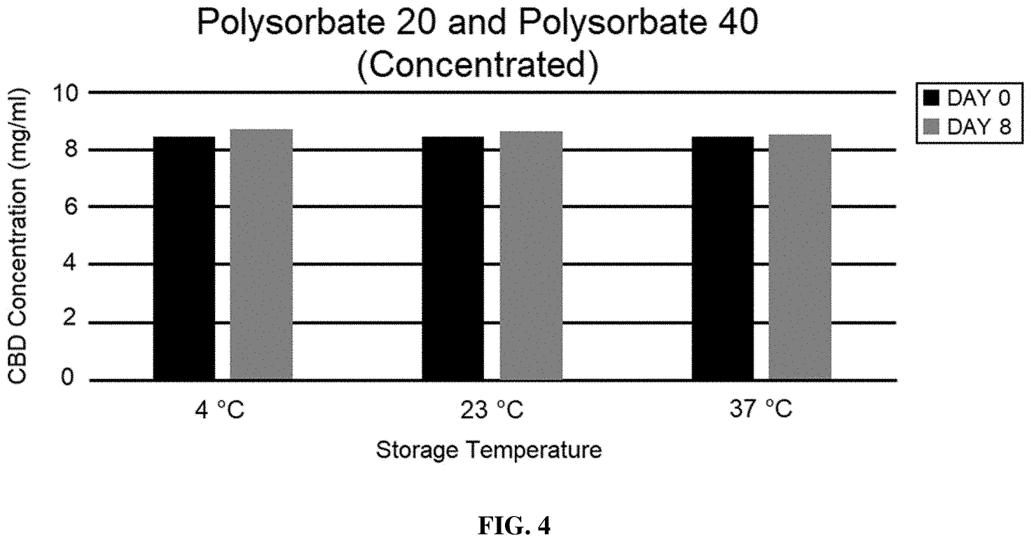

The stock composition from Example 4, containing polysorbate 20, polysorbate 40, vitamin E TPGS, and 8.44 mg/mL of cannabidiol was stored at three separate temperatures of 4° C., room temperature (i.e., about 23° C.), and 37° C. The stock solution at each one of these temperatures was diluted 83.33 fold in drinking water at day 0 and day 8 and analyzed using the HPLC parameters set forth in Example 1. The results are set forth in FIG. 4.

As is apparent from the results set forth in FIG. 4, the concentrated composition containing polysorbate 20, polysorbate 40, vitamin E TPGS, and 8.44 mg/mL of cannabidiol was stable, resulting in minimal loss of cannabidiol at temperatures of 4° C., room temperature (i.e., about 23° C.), and 37° C.

### Example 6

This example illustrates the stability of a diluted composition containing polysorbate 20, polysorbate 40, vitamin E TPGS, and cannabidiol.

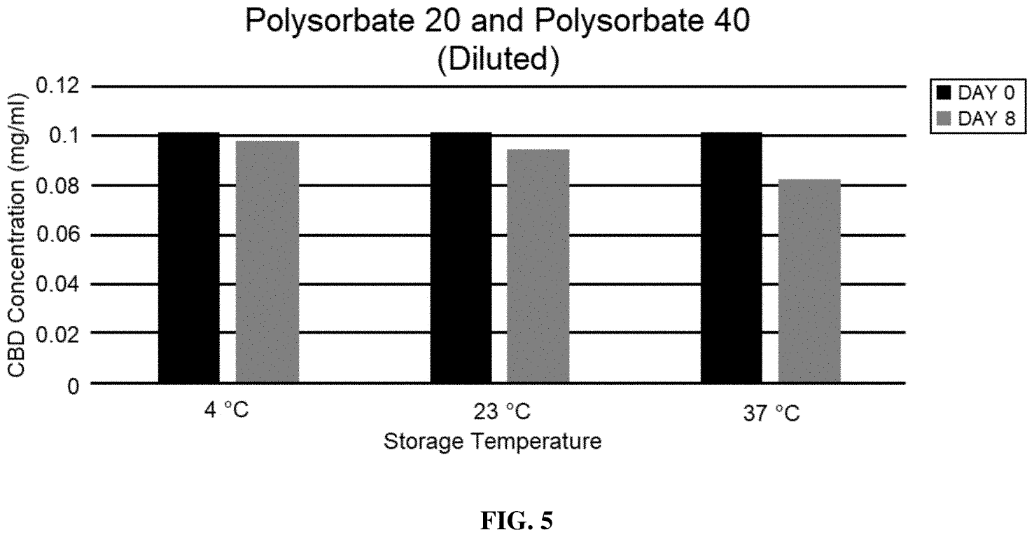

The diluted composition from Example 4, containing polysorbate 20, polysorbate 40, vitamin E TPGS, and 0.101 mg/mL of cannabidiol was stored at three separate temperatures of 4° C., room temperature (i.e., about 23° C.), and 37° C. The diluted composition at each of these temperatures was analyzed at day 0 and day 8 using the HPLC parameters set forth in Example 1. The results are set forth in FIG. 5.

As is apparent from the results set forth in FIG. 5, the diluted composition containing polysorbate 20, polysorbate 40, vitamin E TPGS, and 0.101 mg/mL of cannabidiol maintain comparable stability relative to the concentrated composition of Example 5 at 4° C., resulting in minimal loss of cannabidiol.

### Example 7

This example illustrates a method of preparing a water soluble cannabidiol composition containing polysorbate 20, polysorbate 60, vitamin E TPGS, and cannabidiol.

Polysorbate 20 (7.5 mL) and polysorbate 60 (7.5 mL) were added to high performance liquid chromatography (HPLC) water (985 mL) at 50° C. Vitamin E TPGS (7.5 g) was added to the resulting mixture and the mixture was stirred for 30-60 minutes at 50° C. Cannabidiol (15 g) was slowly added at 50° C. and the resulting composition was mixed for 16 hours. The resulting mixture was filtered through a 0.7 μm Whatman™ glass microfiber filter followed by a 0.2 μm polyethersulfone (PES) filter to produce a composition containing 10.05 mg/mL cannabidiol. The composition was diluted 83.33-fold in water and filtered through a 0.22 μm polyethersulfone (PES) filter to produce a composition containing 0.12 mg/mL cannabidiol. The diluted composition containing 0.12 mg/mL cannabidiol was analyzed using the HPLC parameters set forth in Example 1.

### Example 8

This example illustrates the stability of a concentrated composition containing polysorbate 20, polysorbate 60, vitamin E TPGS, and cannabidiol.

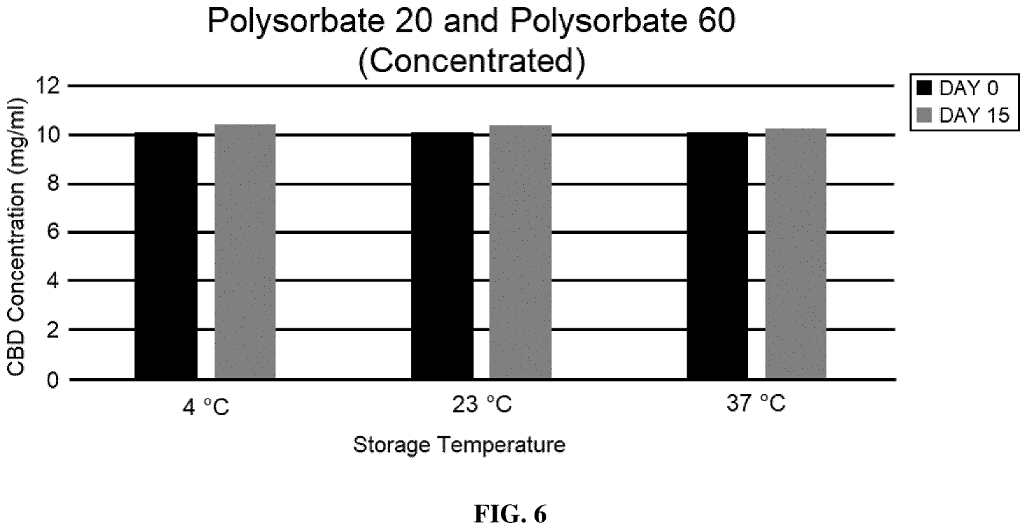

The stock composition from Example 7, containing polysorbate 20, polysorbate 60, vitamin E TPGS, and 10.05 mg/mL of cannabidiol was stored at three separate temperatures of 4° C., room temperature (i.e., about 23° C.), and 37° C. The stock solution at each one of these temperatures was diluted 83.33 fold in drinking water at day 0 and day 15 and analyzed using the HPLC parameters set forth in Example 1. The results are set forth in FIG. 6.

As is apparent from the results set forth in FIG. 6, the concentrated composition containing polysorbate 20, polysorbate 60, vitamin E TPGS, and 10.05 mg/mL of cannabidiol was stable, resulting in minimal loss of cannabidiol at temperatures of 4° C., room temperature (i.e., about 23° C.), and 37° C.

### Example 9

This example illustrates the stability of a diluted composition containing polysorbate 20, polysorbate 60, vitamin E TPGS, and cannabidiol.

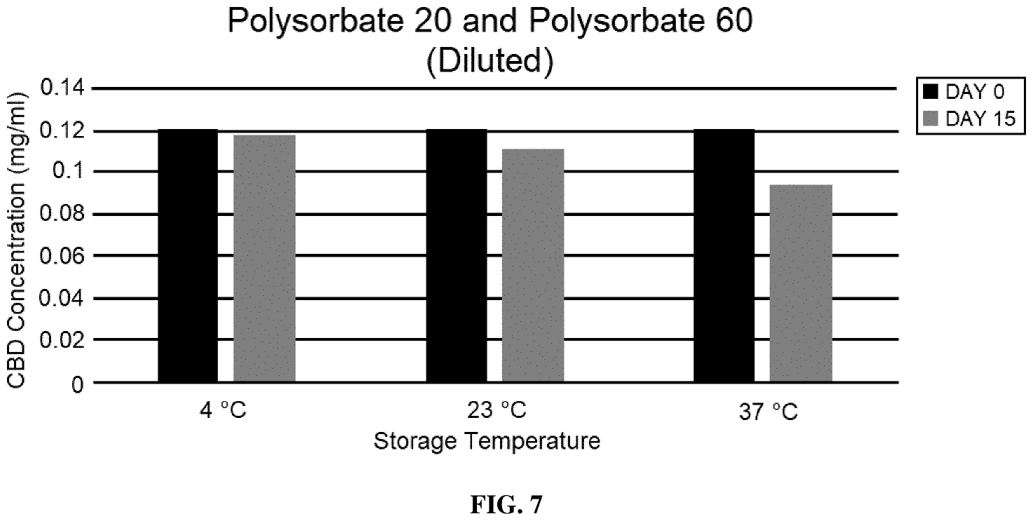

The diluted composition from Example 7, containing polysorbate 20, polysorbate 60, vitamin E TPGS, and 0.12 mg/mL of cannabidiol was stored at three separate temperatures of 4° C., room temperature (i.e., about 23° C.), and 37° C. The diluted composition at each of these temperatures was analyzed at day 0 and day 15 using the HPLC parameters set forth in Example 1. The results are set forth in FIG. 7.

As is apparent from the results set forth in FIG. 7, the diluted composition containing polysorbate 20, polysorbate 60, vitamin E TPGS, and 0.12 mg/mL of cannabidiol maintain comparable stability relative to the concentrated composition of Example 8 at 4° C., resulting in minimal loss of cannabidiol.

### Example 10

This example illustrates a method of preparing a water soluble cannabidiol composition containing polysorbate 20, polysorbate 80, vitamin E TPGS, and cannabidiol.

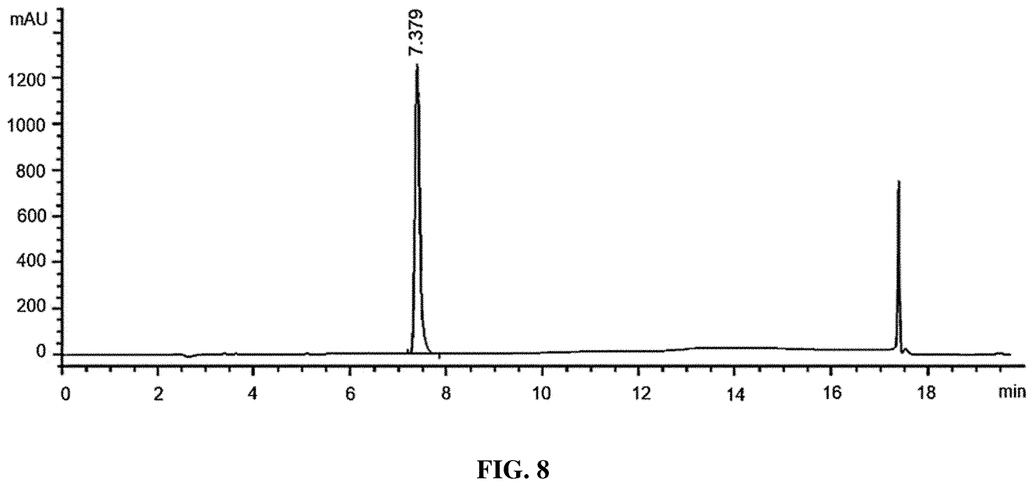

Polysorbate 20 (7.5 mL) and polysorbate 80 (7.5 mL) were added to high performance liquid chromatography (HPLC) water (985 mL) at 50° C. Vitamin E TPGS (7.5 g) was added to the resulting mixture and the mixture was stirred for 30-60 minutes at 50° C. Cannabidiol (15 g) was slowly added at 50° C. and the resulting composition was mixed for 16 hours. The resulting mixture was filtered through a 0.7 μm Whatman™ glass microfiber filter followed by a 0.2 μm polyethersulfone (PES) filter to produce a composition containing 8.41 mg/mL cannabidiol. The composition was diluted 83.33-fold in water and filtered through a 0.22 μm polyethersulfone (PES) filter to produce a composition containing 0.101 mg/mL cannabidiol. The diluted composition containing 0.101 mg/mL cannabidiol was analyzed using the HPLC parameters set forth in Example 1. The resulting HPLC trace is set forth in FIG. 8.

### Example 11

This example illustrates the stability of a concentrated composition containing polysorbate 20, polysorbate 80, vitamin E TPGS, and cannabidiol.

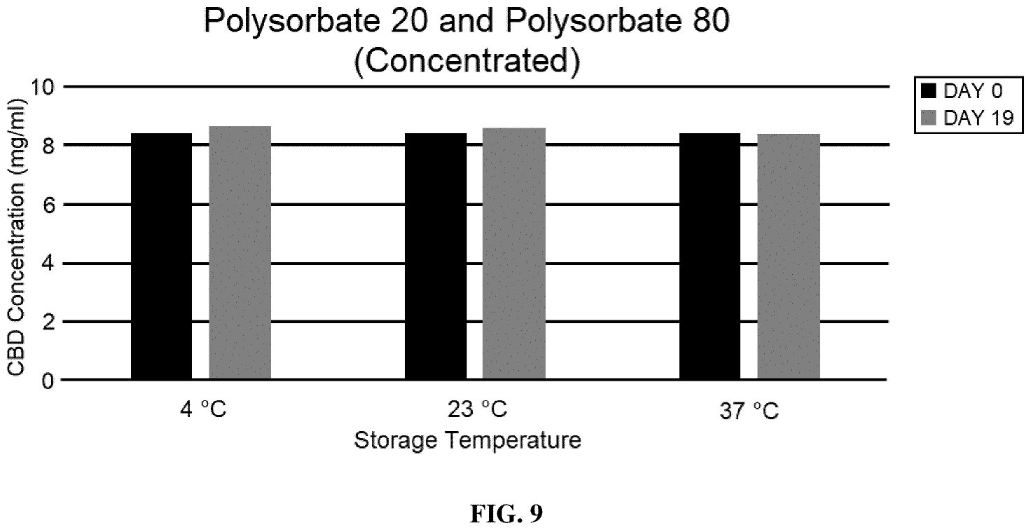

The stock composition from Example 10, containing polysorbate 20, polysorbate 80, vitamin E TPGS, and 8.41 mg/mL of cannabidiol was stored at three separate temperatures of 4° C., room temperature (i.e., about 23° C.), and 37° C. The stock solution at each one of these temperatures was diluted 83.33 fold in drinking water at day 0 and day 19 and analyzed using the HPLC parameters set forth in Example 1. The results are set forth in FIG. 9.

As is apparent from the results set forth in FIG. 9, the concentrated composition containing polysorbate 20, polysorbate 80, vitamin E TPGS, and 8.41 mg/mL of cannabidiol was stable, resulting in minimal loss of cannabidiol at temperatures of 4° C., room temperature (i.e., about 23° C.), and 37° C.

### Example 12

This example illustrates the stability of a diluted composition containing polysorbate 20, polysorbate 80, vitamin E TPGS, and cannabidiol.

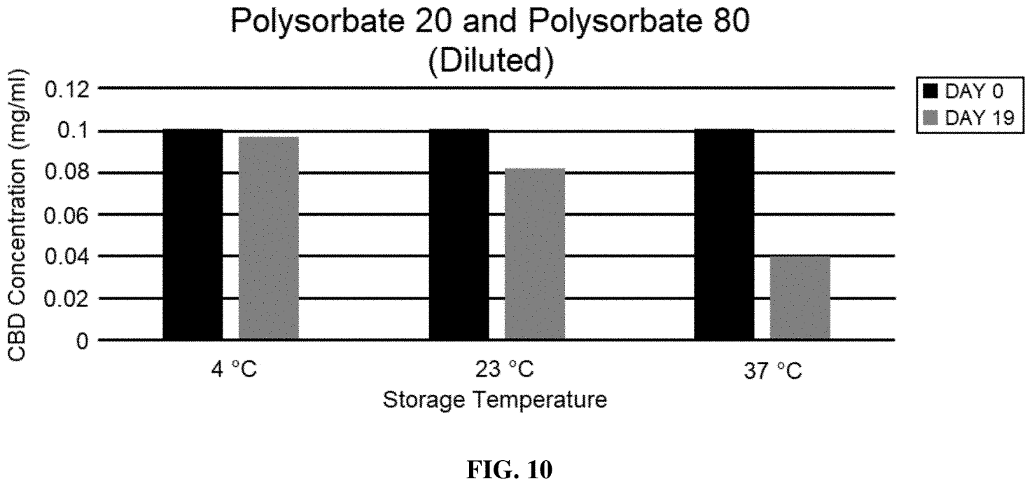

The diluted composition from Example 10, containing polysorbate 20, polysorbate 80, vitamin E TPGS, and 0.101 mg/mL of cannabidiol was stored at three separate temperatures of 4° C., room temperature (i.e., about 23° C.), and 37° C. The diluted composition at each of these temperatures was analyzed at day 0 and day 19 using the HPLC parameters set forth in Example 1. The results are set forth in FIG. 10.

As is apparent from the results set forth in FIG. 10, the diluted composition containing polysorbate 20, polysorbate 80, vitamin E TPGS, and 0.12 mg/mL of cannabidiol maintain comparable stability relative to the concentrated composition of Example 11 at 4° C., resulting in minimal loss of cannabidiol.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments can become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A composition comprising:
(a) a polysorbate,
(b) vitamin E TPGS, and
(c) a cannabinoid.

2. The composition of claim 1, comprising:
(a) from about 30 wt. % to about 50 wt. % of the polysorbate,
(b) from about 10 wt. % to about 30 wt. % of vitamin E TPGS, and
(c) from about 20 wt. % to about 60 wt. % of a cannabinoid,
wherein the weight percentage is based on the sum total of (a), (b), and (c).

3. The composition of claim 2, comprising:
(a) from about 35 wt. % to about 45 wt. % of the polysorbate,
(b) from about 15 wt. % to about 25 wt. % of vitamin E TPGS, and
(c) from about 30 wt. % to about 50 wt. % of a cannabinoid,
wherein the weight percentage is based on the sum total of (a), (b), and (c).

4. The composition of claim 1, wherein the polysorbate is polysorbate 20, polysorbate 40, polysorbate 60, polysorbate 80, or a combination thereof.

5. The composition of claim 1, wherein the polysorbate is polysorbate 20 and one or more of polysorbate 40, polysorbate 60, and polysorbate 80.

6. The composition of claim 1, wherein the polysorbate is polysorbate 20.

7. The composition of claim 1, wherein the polysorbate is polysorbate 40 and one or more of polysorbate 20, polysorbate 60, and polysorbate 80.

8. The composition of claim 1, wherein the polysorbate is polysorbate 40.

9. The composition of claim 1, wherein the cannabinoid is cannabidiol, tetrahydrocannabinol, or a combination thereof.

10. The composition of claim 1, wherein the cannabinoid is cannabidiol.

11. The composition of claim 1, wherein the cannabinoid is tetrahydrocannabinol.

12. The composition of claim 1, wherein the composition is a solid.

13. The composition of claim 1, wherein the composition is an oil.

14. The composition of claim 1, wherein the composition is an aqueous solution.

15. The composition of claim 14, wherein the aqueous solution has a cannabinoid concentration of from about 0.05 mg/mL to about 20 mg/mL.

16. The composition of claim 15, wherein the aqueous solution has a cannabinoid concentration of from about 0.05 mg/mL to about 10 mg/mL.

17. The composition of claim 16, wherein the aqueous solution has a cannabinoid concentration of from about 0.05 mg/mL to about 5 mg/mL.

18. The composition of claim 17, wherein the aqueous solution has a cannabinoid concentration of from about 0.05 mg/mL to about 1 mg/mL.

19. The composition of claim 1, wherein the composition further comprises one or more pharmaceutically acceptable excipients.

20. A method of making a composition for oral consumption comprising:
(a) a polysorbate,
(b) vitamin E TPGS, and
(c) a cannabinoid,
the method comprising:
preparing an aqueous solution comprising the polysorbate, vitamin E TPGS, and the cannabinoid, and
filtering the aqueous solution to form a filtered aqueous solution.

21. The method of claim 20, wherein the polysorbate is polysorbate 20, polysorbate 40, polysorbate 60, polysorbate 80, or a combination thereof.

22. The method of claim 20, wherein the polysorbate is polysorbate 20 and one or more of polysorbate 40, polysorbate 60, and polysorbate 80.

23. The method of claim 20, wherein the polysorbate is polysorbate 20.

24. The method of claim 20, wherein the polysorbate is polysorbate 40 and one or more of polysorbate 20, polysorbate 60, and polysorbate 80.

25. The method of claim 20, wherein the polysorbate is polysorbate 40.

26. The method of claim 20, wherein the cannabinoid is cannabidiol, tetrahydrocannabinol, or a combination thereof.

27. The method of claim 26, wherein the cannabinoid is cannabidiol.

28. The method of claim 26, wherein the cannabinoid is tetrahydrocannabinol.

29. The method of claim 20, further comprising diluting the filtered aqueous solution to form a diluted aqueous solution.

30. The method of claim 20, further comprising removing at least a portion of the water from the filtered aqueous solution to form a concentrated aqueous solution.

31. The method of claim 30, wherein the concentrated aqueous solution is a solid.

32. The method of claim 30, wherein the concentrated aqueous solution is an oil.

33. The method of claim 20, further comprising adding a pharmaceutically acceptable excipient to the aqueous solution, the filtered aqueous solution, the diluted aqueous solution, the concentrated aqueous solution, or a combination thereof.
